(19) United States
(12) Patent Application Publication
(10) Pub. No.: US 2026/0276626 A1
(43) Pub. Date: Sep. 17, 2026

(54) METHOD FOR SCREENING THERAPEUTIC AGENT FOR MUSCLE WEAKNESS-RELATED DISEASES

(71) Applicant: ENCELL CO., LTD., Seoul (KR)

(72) Inventors: Hong Bae Jeon, Seoul (KR); Jangbin Jeong, Seoul (KR); Hyeongseop Kim, Seoul (KR); Taeho Cho, Seoul (KR)

(21) Appl. No.: 19/474,607

(22) PCT Filed: Mar. 12, 2024
(86) PCT No.: PCT/KR2024/003186

§ 371 Date: Oct. 10, 2025

(30) Foreign Application Priority Data
Apr. 12, 2023 (KR) 10-2023-0048087

(51) Int. Cl. G01N 33/50; C12Q 1/6809; C12Q 1/686; C12Q 1/6883

(52) U.S. Cl. CPC G01N 33/5023; C12Q 1/6809; C12Q 1/686; C12Q 1/6883; G01N 33/5044; C12Q 2600/136; G01N 2500/10

(57) ABSTRACT

The present invention relates to a method for screening a therapeutic agent for muscle weakness-related diseases, the method including: (1) a step for treating a myotube with a mitomycin; (2) a step for treating the mitomycin-treated myotube with a candidate substance; and (3) a step for measuring the levels of muscle weakness-related parameters in the cells treated with the candidate substance.

### TECHNICAL FIELD

The present disclosure relates to a method for screening drugs for muscle weakness-related diseases using mitomycin-treated myotubes.

### BACKGROUND ART

Muscles, which account for 40 to 50% of the human body, decrease by about 1% every year after the age of 30, reaching a rapid decline after age 65. As aging progresses, skeletal muscle mass and muscle strength decrease, and functional ability gradually declines, leading to a state in which physical activity is not smooth. As the elderly population increases rapidly due to aging, the number of new patients with diseases related to muscle weakness, including sarcopenia, is rapidly increasing.

Sarcopenia refers to a decrease in muscle strength due to a decrease in skeletal muscle mass during aging. Not only is the reduction in skeletal muscle mass, which is the most prominent characteristic of sarcopenia, but also changes in the type of muscle fiber are observed. For example, type 1 and type 2 decrease at a similar rate with age, whereas when sarcopenia occurs, the thickness of type 2 muscle fiber does not change significantly, but the thickness of type 1 muscle fiber decreases noticeably. In relation to this, the World Health Organization (WHO) assigned a disease code (M63.84) to sarcopenia in 2016, and it is known that about 15% of the population over 60 years of age suffers from sarcopenia.

Such muscle weakness-related diseases including sarcopenia are emerging as a socially important issue, and active research is being conducted thereon. However, because therapeutic agents are insufficient, there is a continuing need for research to develop effective drugs.

Accordingly, the present inventor has developed a novel screening system for selecting a therapeutic agent by modeling a muscle weakness-related disease at a low treatment concentration of mitomycin in a short time compared to a conventional reagent for selecting a therapeutic agent for a muscle weakness-related disease, thereby completing the present disclosure.

### DISCLOSURE

### Technical Problem

An object of the present disclosure is to provide a method for screening a therapeutic agent for a muscle weakness-related disease, comprising: (1) treating myotubes with mitomycin; (2) treating the mitomycin-treated myotubes with a candidate substance; and (3) measuring the levels of muscle weakness-related parameters in the cells (the in-vitro model) treated with the candidate substance.

Another object of the present disclosure is to provide a composition for inducing muscle atrophy and/or sarcopenia comprising mitomycin.

Still another object of the present disclosure is to provide a myotube cultured by treating the myotube with mitomycin in vitro.

Still another object of the present disclosure is to provide a myotube treated with mitomycin in vitro to decrease an expression level of any one or more selected from MyOD, HGF, IGF, MGF, UCP-3, PGC-1α, and myogenin, or to increase an expression level of any one or more selected from MuRF-1, Atrogin-1, TNF-α, IL-6, IL-1β, Caspase-3, and Caspase-8.

### Technical Solution

Each description and embodiment disclosed in the present disclosure may also be applied to each other description and embodiment. In other words, all combinations of various elements disclosed in the present disclosure fall within the scope of the present disclosure. In addition, it may not be considered that the scope of the present disclosure is limited by the detailed description described below.

Hereinafter, in order to prevent congestion of overlapping contents, description of overlapping contents will be omitted. That is, the contents of the invention are not limited only to the following contents, and the contents of the invention should be interpreted according to the overall contents of the invention.

In addition, the terms used in the present specification are used only for the purpose of description and should not be interpreted as limiting. Singular expressions include plural expressions unless the context clearly indicates otherwise. In the present specification, terms such as “comprise” or “have” are intended to designate the presence of features, numbers, steps, operations, components, parts, or combinations thereof described in the specification and it should not be understood as precluding the possibility of the presence or addition of one or more other features, numbers, steps, operations, components, parts, or combinations thereof.

In addition, unless otherwise defined, all terms used herein, including technical or scientific terms, have the same meanings as those generally understood by those of ordinary skill in the art to which the embodiments pertain. It will be further understood that terms, such as those defined in commonly used dictionaries, should be interpreted as having a meaning that is consistent with their meaning in the context of the relevant art, and will not be interpreted in an idealized or overly formal sense unless expressly so defined herein.

Hereinafter, the present disclosure will be described in detail.

The present disclosure provides a method for screening a therapeutic agent for a muscle weakness-related disease, comprising: (1) treating myotubes with mitomycin; (2) treating the mitomycin-treated myotubes with a candidate substance; and (3) measuring the levels of muscle weakness-related parameters in the cells treated with the candidate substance.

As used herein, “muscle weakness” refers to a condition in which one or more muscles have reduced strength or reduced muscle mass. The weakening of muscle strength may be limited to any one muscle, one side of the body, upper or lower limbs, or may appear throughout the body. In addition, subjective muscle weakness symptoms including muscle fatigue or muscle pain may be quantified in an objective manner through medical examination. The causes of muscle weakness may include, but are not limited to, lack of exercise, lack of nutrition, nutritional imbalance, nerve damage, hormone imbalance, decreased ATP production of muscle cells, muscle damage, muscle mass reduction due to decreased differentiation of muscle cells, muscle aging, etc. In addition, it may be a symptom caused by a disease including cachexia, etc.

The muscle weakness-related diseases related to the muscle weakness symptoms may include diseases such as sarcopenia, muscle atrophy, myasthenia, muscular dystrophy, myotonia, hypotonia, muscle weakness, muscle degeneration, etc. Preferably, it may be sarcopenia.

The method for screening a therapeutic agent for a muscle weakness-related disease according to the present disclosure comprises a step of treating myotubes with mitomycin.

In the present disclosure, myotubes refer to cells in which myoblasts are elongated in the process of differentiation of myocytes. Myotubes are characterized by an increase in the number and thickness of muscle fibers, resulting from the fusion between cells. These myotubes show increased expression levels of muscle-specific transcription factors (e.g., myogenin, MyoD, etc.) compared to myoblasts. In addition, the myotubes show lower expression levels of muscle protein degradation-related genes (e.g., MuRF-1 (Muscle RING finger 1), Atrogin-1, FoxO3a, Myostatin, etc.) compared to myoblasts.

The mitomycin may be utilized as a material, a reagent, or a drug for inducing muscle atrophy and/or sarcopenia capable of exhibiting the action of muscle atrophy and/or muscle loss such that a muscle weakness-related disease may be screened.

Treatment of myotubes with mitomycin according to the present disclosure may cause sarcopenia (muscle loss) and/or muscle atrophy with respect to the myotubes. More specifically, treatment with mitomycin may indicate morphological feature changes such as reduction in the number of myotubes, reduction in the diameter of myotubes, reduction in the length of myotubes, reduction in the number of nuclei per myocyte, and apoptosis of myotubes in myotubes. In addition, the expression of genes such as myogenin and MyoD, which are muscle-specific transcription factors involved in myogenic differentiation, may be reduced. Further, the expression levels of muscle protein degradation-related genes, such as Muscle RING finger 1 (MuRF-1), Atrogin-1, Fox03a, and Myostatin, etc., may be increased.

The mitomycin may be mitomycin A, mitomycin B, mitomycin C, etc., and may preferably be mitomycin C.

The mitomycin includes all pharmaceutically acceptable salts thereof, as well as any possible solvates, hydrates, racemates, or stereoisomers capable of being prepared therefrom.

The chemical structure of the mitomycin is shown in the following Chemical Formulas 1 to 3, respectively.

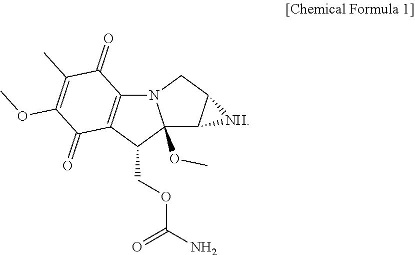 [SMILES: COC1=C(C)C(=O)C2=C(C1=O)[C@@H](COC(N)=O)[C@@]1(OC)[C@H]3N[C@H]3CN21]

The Chemical Formula 1 above represents mitomycin A.

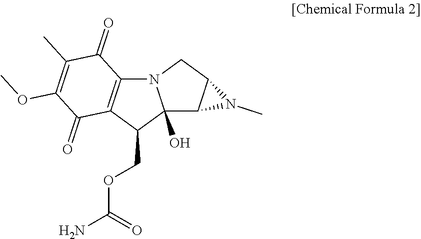

The Chemical Formula 2 above represents mitomycin B.

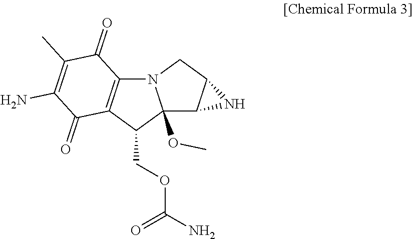 [SMILES: CO[C@@]12[C@H](COC(N)=O)C3=C(C(=O)C(C)=C(N)C3=O)N1C[C@@H]1N[C@@H]12]

The Chemical Formula 3 above represents mitomycin C.

The myotubes according to the present disclosure may be preferably derived from myoblasts. The myoblasts may be obtained from a commercially available cell line, for example, C2C12, C2, Sol8, L6, QM7 (Quail muscle clone 7), L8, H9c2 (2-1), G-7, G-8, and the like, but are not limited thereto. Further, myoblasts from primary cell directly derived from tissues and myoblasts differentiated from pluripotent stem cells (e.g., embryonic stem cells, induced pluripotent stem cells) are also available.

In the present disclosure, myotubes may be prepared by performing the following steps, prior to Step (1):
(a) culturing myoblasts in a culture medium; and
(b) differentiating the cultured myoblasts into myotubes in a differentiation medium.

In an embodiment, the myoblast may be C2C12, and the C2C12 cell line undergoes morphological and genetic changes during myogenesis, which differentiates into myotubes.

As used herein, the term “culture media” refers to a substance capable of supporting the growth and survival of cells in vitro.

The medium may include Dulbecco's Modified Eagle's Medium (DMEM), Minimal Essential Medium (MEM), Basic Medium Eagle (BME), RPMI 1640, F-10, F-12, DMEM/F12, Q-MEM (α-Minimal Essential Medium), G-MEM (Gelgow's Minimal Essential Medium), IMDM (Iscove's Modified Dulbecco's Medium), MacCoy's 5A medium, AmnioMax complete medium, AminoMax±complete medium, EBM (Endothelial Basal Medium) medium, Chang's Medium, MesenCult-XF, DMEM/HG (Dulbecco's Modified Eagle's Medium high glucose) medium, MCDB+DMEM/LG (MCDB+Dulbecco's Modified Eagle's Medium low glucose), etc., but is not particularly limited thereto.

In addition, the medium may include a substance commonly used in the art to prevent the growth of antibiotics, antifungal agents, and/or mycoplasma in order to prevent infection with bacteria, fungi, and the like.

Specifically, the medium may include antibiotics such as penicillin, streptomycin, gentamicin, etc., and preferably penicillin/streptomycin.

In addition, the medium may contain, but is not limited to, commonly used substances such as Amphotericin B as an antifungal agent and gentamicin, ciprofloxacin, and azithromycin as mycoplasma inhibitors.

Further, the medium may include serum isolated from animals such as fetal bovine, calf, horse, sheep, pig, dog, and goat, and preferably may include fetal bovine serum (FBS) and/or horse serum (HS). The content of FBS and/or HS contained in the medium may be 2% to 20%. The medium according to an embodiment of the present disclosure may include 5%, 10%, 15%, or 20% of FBS, and preferably may include 10% of FBS. The medium according to an embodiment of the present disclosure may include 2%, 3%, 4%, 5%, 6%, 8% or 10% of HS, and preferably 5% of HS. Here, % means v/v %.

In an embodiment, the culture medium may include Dulbecco's modified eagle's medium high glucose (DMEM/HG), FBS and penicillin/streptomycin (P/S), preferably, 90% Dulbecco's modified eagle's medium high glucose (DMEM/HG), 10% FBS and 100 unit/ml PS penicillin/streptomycin (P/S).

In an embodiment, the differentiation medium may include Dulbecco's modified eagle's medium high glucose (DMEM/HG), FBS, HS, and penicillin/streptomycin (P/S), preferably, 95% DMEM/HG (Dulbecco's modified eagle's medium high glucose), 5% HS, and 100 unit/ml penicillin/streptomycin (P/S).

In the present disclosure, Step (1) may be performed in a medium containing serum or a serum-free medium.

In the present disclosure, the Step (1) of treating myotubes with mitomycin may be performed in vitro.

In Step (1), the treatment with mitomycin may be performed by treatment with mitomycin at a concentration of 10 to 200 μM for 12 to 48 hours, preferably treatment with mitomycin at a concentration of 15 to 100 μM for 15 to 40 hours, and more preferably treatment with mitomycin at a concentration of 20 to 60 μM for 20 to 30 hours.

The treatment with mitomycin according to the present disclosure rapidly imparts morphological and genetic changes associated with muscle atrophy and/or muscle loss to myotubes at low concentrations, unlike other previously known substances that cause muscle atrophy and/or muscle loss. The morphological and genetic changes include characteristic information related to the above-described muscle atrophy.

In the present disclosure, the myotubes treated with mitomycin according to Step (1) have reduced thickness, length and number of myotube cells compared to a normal control group not treated with mitomycin. A decrease in the thickness, length, and number of myotubes indicates that muscle weakness is induced.

The method for screening a therapeutic agent for a muscle weakness-related disease according to the present disclosure comprises a step of treating the mitomycin-treated myotubes with a candidate substance.

In the present disclosure, the candidate substance refers to a substance expected to promote the differentiation of myoblasts or the formation of myotubes, or increase the expression of genes inducing the differentiation of myoblasts, or inhibit the expression of genes inhibiting differentiation thereof. For example, candidate substances include, but are not limited to, cells, synthetic compounds, natural compounds, low molecular weight compounds, high molecular weight compounds, nucleic acid molecules (e.g., DNA, RNA, PNA and aptamers), proteins, sugars and lipids, and the like.

The candidate substance may be treated for different times and concentrations, etc., depending on the type of the candidate substance described above, the normal treatment concentration of the candidate substance, etc. Preferably, it is preferable that the candidate substance be treated at an appropriate concentration and for an appropriate duration to detect morphological and/or genetic changes in myotubes without affecting cytotoxicity. The appropriate time and concentration for such treatment are obvious to those skilled in the art and may be appropriately adjusted.

The “therapeutic agent” of the present disclosure is a substance that can improve or beneficially alter symptoms (e.g., muscle atrophy and/or muscle loss) caused by muscle weakness-related diseases. Symptoms caused by muscle weakness-related diseases may be improved or beneficially changed by promoting the differentiation of myoblasts or the formation of myotubes to maintain or strengthen muscles.

The method for screening a therapeutic agent for a muscle weakness-related disease according to the present disclosure comprises measuring the levels of muscle weakness-related parameters in the cells treated with the candidate substance.

The muscle weakness-related parameter may be, but is not limited to, an expression level of an oxidative stress marker, an expression level of a muscle cell apoptosis marker, an expression level of a muscle protein synthesis marker, or an expression level of a muscle protein degradation marker.

More specifically, the muscle weakness-related parameter may include at least one selected from the group consisting of glutathione, GPX, SOD, CAT (Catalase), Bax, Bcl-2 family, PARP, caspase, AIF, SIRT, Akt, mTOR, IGF-I, Muscle RING finger 1 (MuRF-1), Atrogin-1, FoxO3a, Myostatin, MyOD, HGF, MGF, UCP-3, PGC-1α, Myogenin, TNF-α, IL-6, and IL-1β.

Specifically, the muscle weakness-related parameter may be one or more selected from the group consisting of glutathione, GPX, SOD, CAT (Catalase), Akt, mTOR, IGF-I, HGF, MGF, UCP-3, and PGC-1α, and a decrease in the expression level thereof may be considered as an induction of muscle atrophy and/or muscle loss.

In addition, the muscle weakness-related parameter may be one or more selected from the group consisting of Bax, Bcl-2 family, PARP, caspase, AIF, SIRT, Muscle RING finger 1 (MuRF-1), Atrogin-1, Fox03a, Myostatin, MyoD, Myogenin, TNF-α, IL-6, and IL-1β, and an increase in the expression level thereof may be considered as an induction of muscle atrophy and/or muscle loss.

In addition, in the muscle weakness-related parameter, the oxidative stress marker may include glutathione, antioxidant enzymes (GPX, GSH, SOD, CAT, etc.), etc.

Specifically, a decrease in glutathione may be considered to induce muscle atrophy and/or muscle loss. In addition, when the expression and/or activity level of glutathione is increased by treatment with a candidate substance, the substance may be selected as a therapeutic candidate substance (therapeutic agent).

Specifically, decrease in the activity levels of Superoxide Dismutase (SOD), Catalase and/or Glutathione peroxidase (GPx) as the antioxidant enzymes may be considered to induce muscle atrophy and/or muscle loss. In addition, when the expressions and/or activity levels of the antioxidant enzymes (Superoxide Dismutase (SOD), Catalase and/or Glutathione peroxidase (GPx)) are increased by treatment with a candidate substance, the substance may be selected as a therapeutic candidate substance (therapeutic agent).

In the muscle weakness-related parameter, the muscle cell apoptosis marker may be at least one selected from the group consisting of Bax, Bcl-2 family, PARP, caspase (for example, caspase-3 and/or caspase-8), AIF, and SIRT, as an apoptosis-related protein and/or gene. Specifically, an increase in any one or more selected from the group consisting of Bax, Bcl-2 family, PARP, caspase, AIF, and SIRT may be considered to induce muscle atrophy and/or muscle loss. In addition, when the expression and/or activity level of any one or more selected from the group consisting of Bax, Bcl-2 family, PARP, caspase, AIF, and SIRT is decreased by treatment with a candidate substance, the substance may be selected as a therapeutic candidate substance (therapeutic agent).

In the muscle weakness-related parameter, the muscle cell apoptosis marker may be TNF-α, IL-1β, and/or IL-6 as a cytokine. Specifically, an increase in TNF-α, IL-1β, and/or IL-6 may be considered to induce muscle atrophy and/or muscle loss. In addition, when the expression and/or activity level of TNF-α, IL-1β, and/or IL-6 is decreased by treatment with a candidate substance, the substance may be selected as a therapeutic candidate substance (therapeutic agent).

In the muscle weakness-related parameter, the muscle cell apoptosis marker may further include a myogenic transcription factor MyoD or myogenin. Specifically, a decrease in MyOD and/or myogenin may be regarded as inducing muscle atrophy and/or muscle loss. In addition, when the expression and/or activity level of MyoD and/or myogenin is increased by treatment with candidate substance, the substance may be selected as a therapeutic candidate substance (therapeutic agent).

In the muscle weakness-related parameter, the muscle protein synthesis marker may be Akt, mTOR, IGF-I, MGF, HGF, UCP-3, and/or PGC-1α. Specifically, reduction or inactivation of Akt, mTOR, IGF-I, MGF, HGF, UCP-3, and/or PGC-1α may be regarded as inducing muscle atrophy and/or muscle loss. Further, when Akt, mTOR, IGF-I, MGF, HGF, UCP-3, and/or PGC-1α are increased, phosphorylated or activated by treatment with a candidate substance, the substance may be selected as a therapeutic candidate substance (therapeutic agent).

In the muscle weakness-related parameter, the muscle protein degradation marker may include Muscle RING finger 1 (MuRF-1), Atrogin-1, FoxO3a, Myostatin, and the like. Specifically, an increase in Muscle RING finger 1 (MuRF-1), Atrogin-1, FoxO3a and/or myostatin may be regarded as inducing muscle atrophy and/or muscle loss. In addition, when Muscle RING finger 1 (MuRF-1), Atrogin-1, FoxO3a and/or myostatin are reduced or not activated by treatment with a candidate substance, the substance may be selected as a therapeutic candidate substance (therapeutic agent).

In the present disclosure, preferably, the marker may be any one or more selected from the group consisting of Muscle RING finger 1 (MuRF-1), Atrogin-1, FoxO3a, and myostatin.

An increase expression level of any one or more markers selected from the group consisting of MuRF-1, Atrogin-1, FoxO3a, and myostatin may be considered as an induction of muscle atrophy and/or muscle loss.

In the present disclosure, the myotubes treated with mitomycin may have an increased expression level of any one or more selected from the group consisting of MuRF-1, atrogin-1 (atrogin-1), FoxO3a, and myostatin, compared to a normal control group not treated with mitomycin.

In addition, when the level of any one or more markers selected from the group consisting of MuRF-1, Atrogin-1, FoxO3a, and myostatin is reduced by treatment with a candidate substance, the substance may be selected as a therapeutic candidate substance (therapeutic agent).

More preferably, the muscle weakness-related parameter may be MuRF-1 and/or Atrogin-1. Unlike general ubiquitin ligases in the body that induce unnecessary protein apoptosis, the Atrogin-1 and MuRF-1 show an over-expression pattern mainly in patients with muscular diseases accompanied by pathological muscle loss, loss of muscle mass, or muscle atrophy, and are currently known as one of the direct molecular biological causes of muscle weakness-related diseases, particularly sarcopenia. When the atrogin-1 and MuRF-1 are overexpressed, a degradation pathway is excessively activated compared to the synthesis of muscle cells, and thus a loss of normal muscle cells occurs.

The expression level of the gene, the amount of the protein, or the activity of the protein may be measured in the cells treated with the candidate substance, and if the measurement results show that the expression level of the gene, the amount of protein, or the activity of the protein increases or decreases, the candidate substance may be determined to be a substance capable of preventing and treating the muscle weakness-related diseases.

When the candidate substance inhibits the expression of the atrogin-1 gene and/or the MuRF-1 gene or inhibits the function or activity of the atrogin-1 protein and/or the MuRF-1 protein, the candidate substance may be selected as a therapeutic agent for preventing or treating the muscle weakness-related diseases.

The screening model using mitomycin according to the present disclosure may greatly reduce the time to induce the muscle atrophy and/or muscle loss and the concentration of the muscle atrophy-inducing agent compared to using the conventionally known muscle atrophy and/or muscle loss inducing substance. Furthermore, the use of a small concentration allows for the use of less solvent, such as dimethyl sulfoxide (DMSO), as a diluent when adding substances, which has the advantage of providing a model more suitable for drug screening without the influence of cytotoxicity, etc.

In the present disclosure, the measuring of the levels of the muscle weakness-related parameters in Step (3) may be preferably to measure the expression level of a gene, the amount of a protein, or the activity of a protein. The measuring may be performed by, but is not limited to, reverse transcription polymerase chain reaction (RT-PCR), enzyme-linked immunosorbent assay (ELISA), western blot, flow cytometry (FACS), or whole cell patch clamp assay.

In addition, the expression level of the oxidative stress marker may be measured using a commercial kit or a spectrophotometer.

The expression level of the muscle cell apoptosis marker, the expression level of the muscle protein synthesis marker, or the expression level of the muscle protein degradation marker may be measured using the commercial kit, or any method capable of measuring the expression level of the protein or gene level described above may be used. For example, the expression level thereof may be measured by, but are not limited to, reverse transcription polymerase chain reaction (RT-PCR), radioimmunoassay, enzyme immunoassay (ELISA), western blot, flow cytometry (FACS), whole cell patch clamp assay, or the like.

In the present disclosure, the method may further comprise (4) selecting a candidate substance that increases or decreases the level of muscle weakness-related parameter in cells treated with the candidate substance, as compared to a control group not treated with the candidate substance.

In the cells treated with the candidate substance, when the expression level of glutathione, which is an oxidative stress marker, among the muscle weakness-related parameters, is increased compared to that of a control group not treated with the candidate substance, the substance may be selected as a therapeutic candidate substance (therapeutic agent).

In addition, in the cells treated with the candidate substance, when the expression and/or activity level of antioxidant stress markers (superoxide dismutase (SOD), Catalase, and/or Glutathione peroxidase (GPx)) among the muscle weakness-related parameters is increased compared to a control group not treated with the candidate substance, the substance may be selected as a therapeutic candidate substance (therapeutic agent).

Further, in the cells treated with the candidate substance, when the expression level of any one or more selected from the group consisting of Bax, Bcl-2 family, PARP, caspase (e.g., caspase-3 or caspase-8), AIF, and SIRT, which are muscle cell apoptosis markers, among the muscle weakness-related parameters, is reduced compared to a control group not treated with the candidate substance, the candidate substance may be selected as a therapeutic candidate substance (therapeutic agent).

In addition, in the cells treated with the candidate substance, when the levels of cytokines TNF-α, IL-1B, and/or IL-6, which are muscle cell apoptosis markers, among the muscle weakness-related parameters, are reduced compared to a control group that is not treated with the candidate substance, the candidate substance may be selected as a therapeutic candidate substance.

Further, in the cells treated with the candidate substance, when the level of MyoD and/or myogenin, which are myogenic transcription factors as the muscle cell apoptosis markers, among the muscle weakness-related parameters, is increased compared to a control group not treated with the candidate substance, the candidate substance may be selected as a therapeutic candidate substance (therapeutic agent).

In addition, in the cells treated with the candidate substance, when the levels of Akt, mTOR, IGF-I, MGF, HGF, UCP-3, and/or PGC-1a, which are muscle protein synthesis markers, are increased, phosphorylated or activated compared to a control group not treated with the candidate substance, the candidate substance may be selected as a therapeutic candidate substance (therapeutic agent).

Further, in the cells treated with the candidate substance, when the level of any one or more selected from the group consisting of muscle protein degradation markers MuRF-1, Atrogin-1, FoxO3a, and myostatin is reduced compared to a control group not treated with the candidate substance, the candidate substance may be selected as a therapeutic candidate substance (therapeutic agent).

In the present disclosure, the muscle weakness-related disease may include sarcopenia, muscle atrophy, myasthenia, muscular dystrophy, myotonia, hypotonia, muscle weakness, or muscle degeneration, and preferably sarcopenia.

In another general aspect, the present disclosure also provides a composition for inducing muscle atrophy and/or muscle loss comprising mitomycin.

The composition for inducing muscle atrophy and/or muscle loss comprising mitomycin according to the present disclosure may be preferably intended for treatment in vitro. In other words, the present disclosure provides a composition for inducing muscle atrophy and/or muscle loss of myotubes in vitro, comprising mitomycin.

The mitomycin according to the present disclosure may cause morphological and genetic changes related to muscle atrophy and/or muscle loss in myotubes. More specifically, the mitomycin may represent changes in morphological characteristics in myotubes, such as a decrease in the number of myotubes, a decrease in the diameter of the myotubes, a decrease in the length of the myotubes, a decrease in the number of nuclei per myocyte, and apoptosis of the myotubes. In addition, the expression of genes such as myogenin and MyOD, which are muscle-specific transcription factors involved in myogenic differentiation, may be reduced. Further, genes related to muscle protein degradation, such as MuRF-1 (Muscle RING finger 1), Atrogin-1, FoxO3a, and Myostatin, may show increased expression levels.

The present disclosure provides a myotube cultured by treating the myotube with mitomycin in vitro. The present disclosure provides a myotube treated with mitomycin in vitro to decrease an expression level of any one or more selected from MyOD, HGF, IGF, MGF, UCP-3, PGC-1a, and myogenin, or to increase an expression level of any one or more selected from MuRF-1, Atrogin-1, TNF-α, IL-6, IL-1β, Caspase-3, and Caspase-8.

More particularly, provided is a myotube treated with mitomycin in vitro to decrease an expression level of any one of more selected from MyOD, HGF, IGF, MGF, UCP-3, PGC-1α, and myogenin.

Further, provided is a myotube treated with mitomycin in vitro to increase an expression level of any one or more selected from MuRF-1, Atrogin-1, TNF-α, IL-6, IL-1β, Caspase-3, and Caspase-8.

The mitomycin-treated myotube is a model for screening a therapeutic agent for a muscle weakness-related disease, and is effective in screening a candidate substance (drug) for preventing or treating the muscle weakness-related disease in vitro.

### Advantageous Effects

The method for screening a therapeutic agent for muscle weakness-related diseases using mitomycin according to the present disclosure may enable a simple and efficient in vitro screening of the therapeutic agent for muscle weakness-related diseases. In particular, the therapeutic agent for muscle weakness-related diseases may be rapidly screened while maintaining a state of cells suitable for modeling using a small amount of reagent, and thus the present disclosure is useful for screening candidate substances.

### DESCRIPTION OF DRAWINGS

FIG. 1 is a schematic diagram showing the process and cell shape in which myoblasts are differentiated into myotubes through myocytes.

FIG. 2 shows the morphological changes in myotubes from the start of differentiation to Day 5 in the myotube differentiation process according to Example 1.

FIG. 3 shows the mRNA expression levels of MuRF-1 and Atrogin-1 in myoblasts and myotubes.

FIG. 4 shows the morphological changes in myotubes according to the treatment concentration and treatment time of muscle atrophy and/or sarcopenia-inducing drugs.

FIG. 5 shows the changes in the mRNA expression levels of MuRF-1 and Atrogin-1 in myotubes according to the treatment concentration and treatment time of muscle atrophy and/or muscle loss-inducing drugs.

FIG. 6 shows the morphological changes in myotubes treated with muscle atrophy and/or muscle loss-inducing drugs in serum-free medium.

FIG. 7 shows the changes in the mRNA expression levels of MuRF-1 and Atrogin-1 in myotubes treated with muscle atrophy and/or muscle loss-inducing drugs in serum-free medium.

FIG. 8 shows the morphological changes in myotubes when treating a muscle weakness-related disease screening model prepared according to Example 1 with a therapeutic agent for muscle weakness-related diseases.

FIG. 9 shows the changes in the mRNA expression levels of MuRF-1 and Atrogin-1 in myotubes when treating a muscle weakness-related disease screening model prepared according to Example 1 with a therapeutic agent for muscle weakness-related diseases.

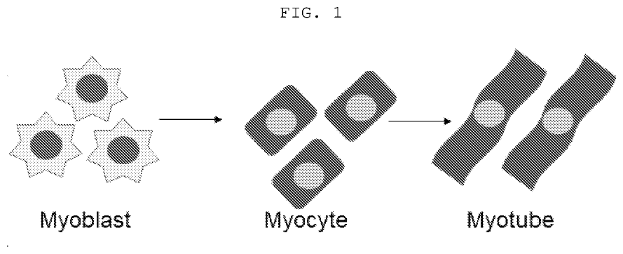

### BEST MODE

Hereinafter, preferred examples are presented to aid in understanding of the present disclosure. However, the following Examples are provided only to more easily understand the present disclosure, and the contents of the present disclosure are not limited by these Examples.

In the present disclosure, Dulbecco's modified eagle's medium (DMEM) high glucose used for cell culture and cell differentiation was purchased from Biowest, fetal bovine serum (FBS), horse serum (HS), and penicillin/streptomycin (P/S) were purchased from Gibco, and Dexamethasone (Dex) and Mitomycin C from Streptomyces caespitosus (MMC) used as reagents for inducing sarcopenia were purchased from Sigma-Aldrich Chemical Co.

### Example 1. Preparation of Muscle Weakness-Related Disease Screening Model

(1) Cell Culture and Differentiation into Myotubes

The C2C12 mouse myoblast cell line was cultured in a culture medium containing 90% DMEM/HG, 10% fetal bovine serum (FBS), and 100 unit/ml penicillin/streptomycin at 37° C. under 5% CO₂ condition.

100,000 cells were seeded in 6-well plate. After 4 days, the medium was replaced with a differentiation medium containing 5% horse serum and 100 unit/ml penicillin/streptomycin, and 95% DMEM/HG for differentiation into myotubes at 100% confluence. Here, the date on which the culture medium was replaced with the differentiation medium was designated as day 0, and the differentiation medium was replaced with fresh differentiation medium every day from day 3 to day 5.

(2) Preparation of Myotubes in which Muscle Atrophy and/or Sarcopenia is Induced

The medium was removed on day 5 to induce muscle atrophy and/or muscle loss, and the culture medium was treated with mitomycin C (hereinafter referred to as MMC) at concentrations of 20 μM, 40 μM, and 100 μM for 24 hours, respectively. An optical microscopic observation was used to identify changes that occurred during the process of muscle atrophy and/or muscle loss. After a predetermined period of time, the medium was removed, and the cells were treated with 0.25% trypsin-ethylene diamine tetra-acetic acid (EDTA, Gibco), and the cells were collected in a culture medium, followed by centrifugation at 1,500 rpm for 3 minutes. Then, the supernatant was removed, and the obtained cells were washed with Dulbecco's phosphate-buffered saline (DPBS, Celgene). After centrifugation at 1500 rpm for 3 minutes, the supernatant was removed and cells were obtained in which muscle atrophy and/or muscle loss was induced.

### (3) Confirmation of MuRF-1 and Atrogin-1 Levels

RNA was extracted from the cells using AccuPrep®Universal RNA Extraction Kit (Bioneer). CDNA was synthesized using SuperScript™ IV Reverse Transcriptase purchased from Invitrogen, and quantitative real-time polymerase chain reaction (qPCR) was performed using PowerSYBR green PCR Master Mix from Applied biosystem to confirm the mRNA expression level.

Primers used in the PCR analysis are shown in Table 1:

TABLE 1

| SEQ | | |
|---|---|---|
| ID NO: | Primer | Sequence |
| 1 | MuRF-1 | GGAGAAGCTGGACTTCATCG |
| | Forward | (SEQ ID NO: 1) |
| 2 | MuRF-1 | CTTGGCACTCAAGAGGAAGG |
| | Reverse | (SEQ ID NO: 2) |
| 3 | Atrogin-1 | GAACATCATGCAGAGGCTGA |
| | Forward | (SEQ ID NO: 3) |
| 4 | Atrogin-1 | GTAGCCGGTCTTCACTGAGC |
| | Reverse | (SEQ ID NO: 4) |

### Comparative Example 1. Preparation of Dexamethasone-Induced Muscle Weakness-Related Disease Screening Model

A muscle weakness-related disease screening model was prepared in the same manner as in Example 1, except that dexamethasone (hereinafter, DEX) at concentrations of 20 UM, 40 μM, and 100 μM was used instead of mitomycin C as an inducer for muscle atrophy and/or muscle weakness.

### Example 2. Confirmation of Differentiation into Myotubes

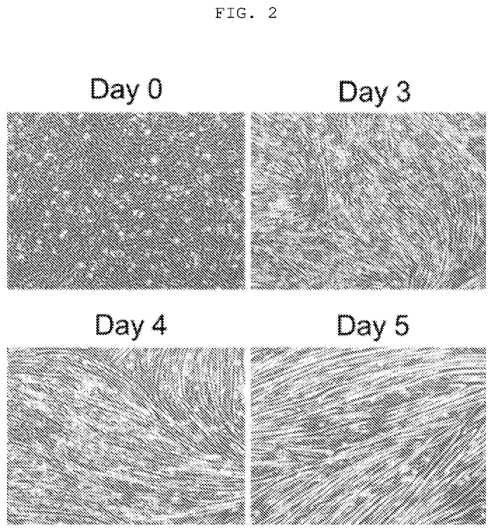

In Example 1, in order to confirm differentiation from myoblasts to myotubes, after induction of differentiation, the thickness and length of the myotubes were confirmed through an optical microscopic observation at random positions in the culture vessel, and the results are as shown in FIG. 2.

As could be seen from FIG. 2, the differentiation into myotubes Day 3 after was confirmed from differentiation was induced by treating C2C12 myoblasts with differentiation medium, and on Day 5, most of the entire cell culture area was differentiated into myotubes. The myotubes observed on Day 5 were thicker, longer, and more numerous than the myotubes observed on Day 3. These results confirmed that differentiation from myoblasts to myotubes was successfully performed.

### Example 3. Determination of Expression Levels of MuRF-1 and Atrogin-1 in Myoblasts and Differentiated Myotubes

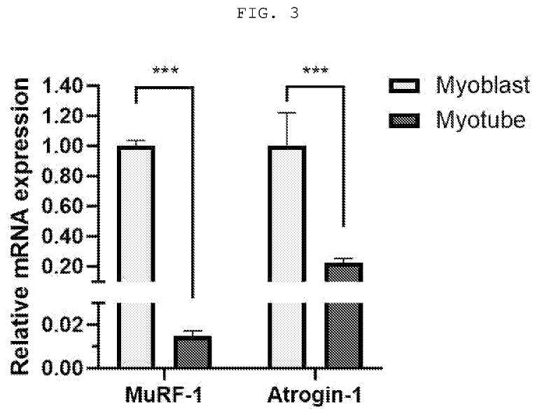

Changes in the expression of the MuRF-1 and Atrogin-1 genes were measured as in Example 1 of the present disclosure, and the results are shown in FIG. 3.

As could be seen in FIG. 3, it was found that the mRNA expression levels of MuRF-1 and Atrogin-1 were reduced completion of differentiation in the myotubes after compared to the myoblasts before differentiation. These results confirmed that differentiation into myotubes was successful.

Through the results of Example 2 and Example 3, it was confirmed that the differentiation into myotubes was well achieved morphologically and genetically.

### Example 4. Confirmation of Effect of Inducing Muscle Atrophy and/or Muscle Loss According to Type and Treatment Concentration of Drugs that Induce Muscle Atrophy and/or Muscle Loss

In order to confirm the effect of inducing muscle atrophy and/or muscle loss according to the type and treatment concentration of the drug inducing muscle atrophy and/or muscle loss, changes in cell morphology, and changes in the mRNA expression levels of MuRF-1 and Atrogin-1 in myotubes treated with mitomycin C at a concentration of 20 μM and 40 μM for 24 hours (Mitomycin C), myotubes (Dexamethasone) treated with dexamethasone at a concentration of 100 μM for 24 hours, and control group (Ctrl) not treated with the drug inducing muscle atrophy and/or muscle loss were confirmed.

### (1) Confirmation of Morphological Changes in Cells

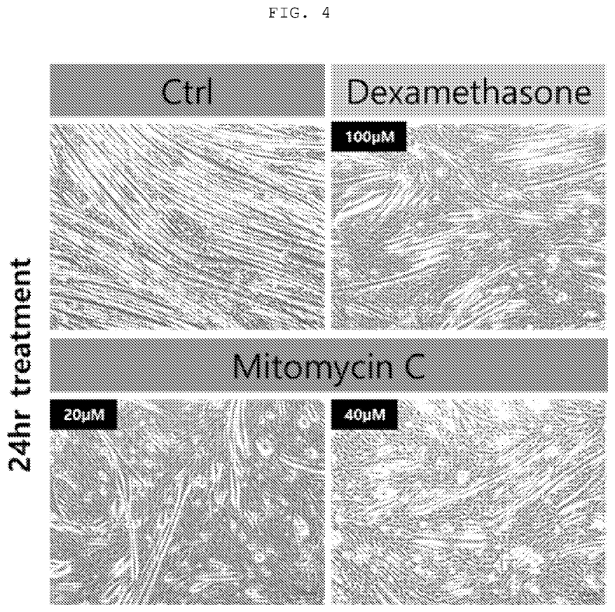

Morphological changes in myotubes according to the type and treatment concentration of drugs that induce muscle atrophy and/or muscle loss were confirmed, and the results are shown in FIG. 4.

As could be seen in FIG. 4, the thickness, length, and number of myotubes were significantly reduced during the 24-hour treatment even though the myotubes were treated with mitomycin C at a relatively low concentration of 40 μM or less. On the other hand, it was confirmed that even when the myotubes were treated with dexamethasone at a high concentration of 100 μM for the same time, muscle atrophy and/or muscle loss were not effectively induced. Moreover, the experimental group treated with a concentration of 100 μM or less did not show significant morphological changes related to sarcopenia.

Therefore, it was confirmed that the use of mitomycin C can induce muscle atrophy and/or muscle loss more rapidly even at a lower concentration.

### (2) Confirmation of Changes in Expression Level of Muscle Weakness-Related Parameter

Changes in mRNA expression of MuRF-1 and Atrogin-1 in myotubes according to the type and treatment concentration of muscle atrophy and/or muscle loss-inducing drugs were confirmed, and the results are shown in FIG. 5.

As could be seen in FIG. 5, the expression levels of MuRF-1 and Atrogin-1 genes were the highest in myotubes treated with mitomycin C at a concentration of 40 μM for 24 hours, which were higher than the mRNA expression levels of MuRF-1 and Atrogin-1 in myotubes treated with dexamethasone at a concentration of 100 μM for 24 hours.

These results indicate that the use of mitomycin C in the production of a muscle weakness-related disease model can produce a model capable of screening for a treatment agent for the muscle weakness-related disease in a simple and efficient manner in vitro by reducing the concentration and time compared to using existing drugs.

(3) Determination of the Effect of Serum-Free State on Induction of Muscle Atrophy and/or Muscle Loss

In using cells induced with muscle atrophy and/or muscle loss as a screening system for the development of therapeutic agents, it was confirmed whether serum free (SF) conditions affect induction of muscle atrophy and/or muscle loss. When serum is included, the effect of sarcopenia may be alleviated due to the provision of cell nutrients and when co-cultured with stem cells, the observed effects may be attributed to the serum rather than to the stem cells alone, and thus muscle atrophy and/or muscle loss was induced in a serum-free medium.

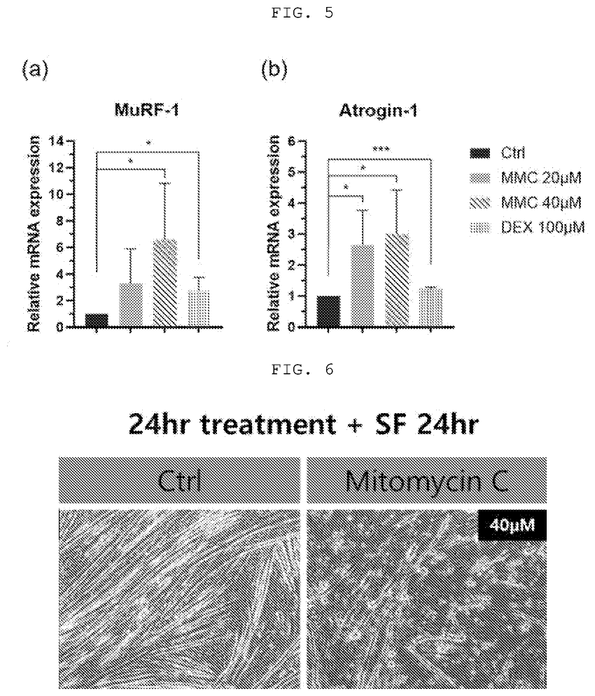

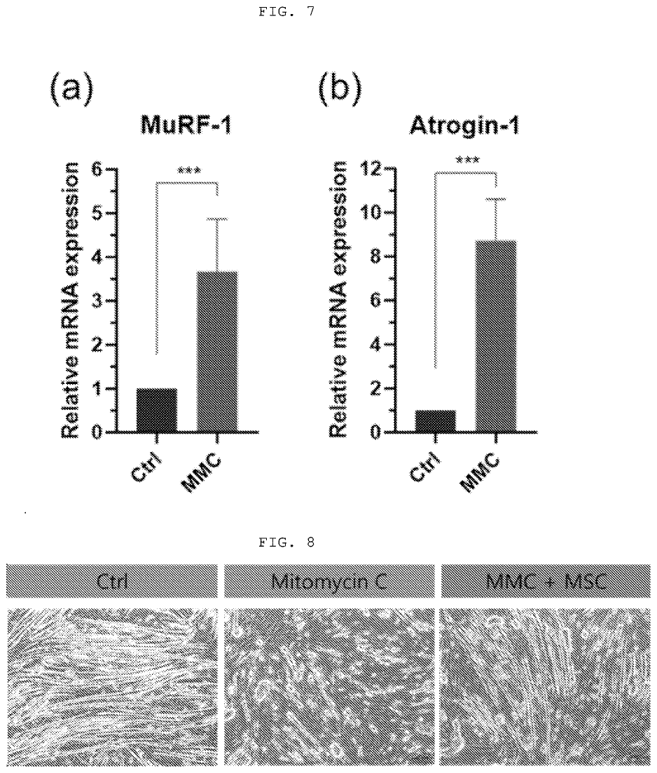

Specifically, myotubes treated with mitomycin C at a concentration of 40 μM for 24 hours (Mitomycin C) and a control (Ctrl) were cultured in a serum-free medium for 24 hours, respectively. Then, the cells were confirmed through optical microscopic observation, and the expression levels of the MuRF-1 and Atrogin-1 genes were confirmed. The results are shown in FIGS. 6 and 7, respectively.

As could be seen in FIGS. 6 and 7, even in the serum-free state, the mitomycin C-treated group showed a significant decrease in the thickness, length, and number of myotubes at relatively low concentrations and for a short period of time, and it was shown that the mRNA expression levels of MuRF-1 and Atrogin-1 were maintained in an increased state.

These results indicate that the serum-free state had no effect on the cells, and a system for screening a therapeutic agent for a muscle weakness-related disease could be efficiently implemented using mitomycin C.

### Example 5. Confirmation of MSC Treatment Efficacy Using Screening Model in which Muscle Atrophy and/or Muscle Loss is Induced

In order to confirm whether the cells induced with muscle atrophy and/or muscle loss according to the present disclosure can be used as a screening system for the development of therapeutic agents, mesenchymal stem cells (MSCs) known to have a recovery effect on muscle atrophy and/or muscle loss were co-cultured with the cells induced with muscle atrophy and/or muscle loss prepared according to Example 1, and then changes in myotube morphology were observed under a microscope and changes in mRNA expression of MuRF-1 and Atrogin-1 were confirmed.

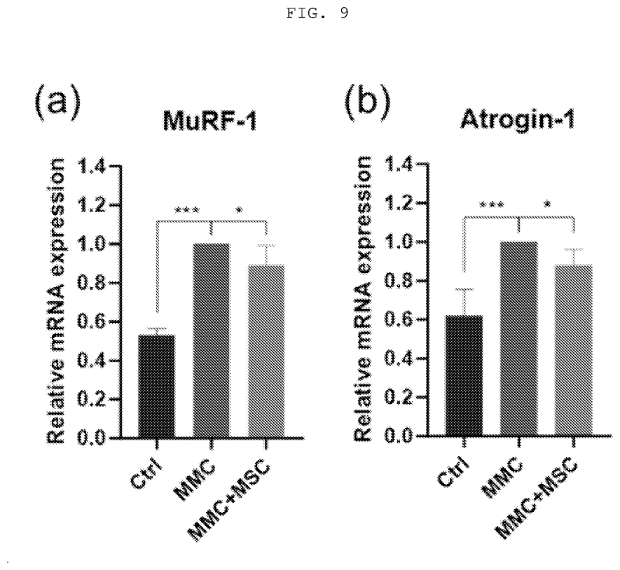

The results are shown in FIGS. 8 and 9, respectively.

As could be confirmed in FIGS. 8 and 9, it was found that the thickness, length and number of myotubes, which were reduced in cells induced with muscle atrophy and/or muscle loss prepared according to Example 1 treated with mitomycin C (MCC), were recovered in the MSC co-culture group (MMC+MSC). In addition, it was observed that the mRNA expression levels of MuRF-1 and Atrogin-1, which were increased in the cells (MMC) induced with muscle atrophy and/or muscle loss prepared according to Example 1, decreased in the MSC co-culture group (MMC+MSC).

These results showed that the screening model for muscle weakness-related diseases according to the present disclosure can be usefully employed for discovering a therapeutic agent for muscle weakness-related diseases.

1. A method for screening a therapeutic agent for a muscle weakness-related disease, comprising:
(1) treating myotubes with mitomycin;
(2) treating the mitomycin-treated myotubes with a candidate substance; and
(3) measuring the levels of muscle weakness-related parameters in the cells (the in-vitro model) treated with the candidate substance.

2. The method of claim 1, wherein the mitomycin is mitomycin A, mitomycin B, or mitomycin C.

3. The method of claim 1, wherein the muscle weakness-related parameter comprises at least one selected from the group consisting of an expression level of an oxidative stress marker, an expression level of a muscle cell apoptosis marker, an expression level of a muscle protein synthesis marker, and an expression level of a muscle protein degradation marker.

4. The method of claim 1, wherein the muscle weakness-related parameter comprises at least one selected from the group consisting of glutathione, GPX, SOD, CAT (Catalase), Bax, Bcl-2 family, PARP, caspase, AIF, SIRT, Akt, mTOR, IGF-I, Muscle RING finger 1 (MuRF-1), Atrogin-1, FoxO3a, Myostatin, MyOD, HGF, MGF, UCP-3, PGC-1α, Myogenin, TNF-α, IL-6, and IL-1B.

5. The method of claim 4, wherein the muscle weakness-related parameter is at least one selected from the group consisting of glutathione, GPX, SOD, CAT (Catalase), Akt, mTOR, IGF-I, HGF, MGF, UCP-3, and PGC-1α, and
when the expression level thereof decreases, muscle atrophy and/or sarcopenia are induced.

6. The method of claim 4, wherein the muscle weakness-related parameter is at least one selected from the group consisting of Bax, Bcl-2 family, PARP, caspase, AIF, SIRT, Muscle RING finger 1 (MuRF-1), Atrogin-1, FoxO3a, Myostatin, MyoD, Myogenin, TNF-α, IL-6, and IL-13, and
when the expression level thereof increases, muscle atrophy and/or muscle loss are induced.

7. The method of claim 1, further comprising, prior to Step (1),
(a) culturing myoblasts in a culture medium; and
(b) differentiating the cultured myoblasts into myotubes in a differentiation medium.

8. The method of claim 1, wherein the step of treating myotubes with mitomycin is performed in vitro.

9. The method of claim 1, further comprising:
(4) selecting a candidate substance that increases or decreases the level of muscle weakness-related parameter in cells treated with the candidate substance, as compared to a control group not treated with the candidate substance.

10. The method of claim 1, wherein in Step (1), the myotubes are treated with mitomycin at a concentration of 10 to 200 μM for 12 to 48 hours.

11. The method of claim 1, wherein the myotubes treated with mitomycin have a reduced thickness, length, and number of myotubes, as compared to a normal control group not treated with mitomycin.

12. The method of claim 1, wherein the myotubes treated with mitomycin have an increased expression level of at least one selected from the group consisting of MuRF-1, Atrogin-1, FoxO3a, and myostatin, as compared to a normal control group not treated with mitomycin.

13. The method of claim 1, wherein the muscle weakness-related disease is any one selected from the group consisting of sarcopenia, muscle atrophy, myasthenia, muscular dystrophy, myotonia, hypotonia, muscle weakness, and muscle degeneration.

14. The method of claim 13, wherein the muscle weakness-related disease is sarcopenia.

15. The method of claim 1, wherein the measuring of the levels of the muscle weakness-related parameters in Step (3) is performed by any one selected from the group consisting of reverse transcription polymerase chain reaction (RT-PCR), enzyme immunoassay (ELISA), immunohistochemistry (IHC), western blot, flow cytometry (FACS), and whole cell patch clamp assay.

16. A composition for inducing muscle atrophy and/or sarcopenia comprising mitomycin.

17. A myotube treated with mitomycin in vitro to decrease an expression level of any one or more selected from the group consisting of MyOD, HGF, IGF, MGF, UCP-3, PGC-1, and myogenin, or to increase an expression level of any one or more selected from the group consisting of MuRF-1, Atrogin-1, TNF-α, IL-6, IL-13, Caspase-3, and Caspase-8.


SEQUENCE LISTING (4 sequences)

SEQ ID NO: 1 - DNA, 20 bp, synthetic construct
  source 1..20: mol_type = other DNA
ggagaagctggacttcatcg

SEQ ID NO: 2 - DNA, 20 bp, synthetic construct
  source 1..20: mol_type = other DNA
cttggcactcaagaggaagg

SEQ ID NO: 3 - DNA, 20 bp, synthetic construct
  source 1..20: mol_type = other DNA
gaacatcatgcagaggctga

SEQ ID NO: 4 - DNA, 20 bp, synthetic construct
  source 1..20: mol_type = other DNA
gtagccggtcttcactgagc